(19) United States
(12) Patent Application Publication
(10) Pub. No.: US 2020/0247959 A1
(43) Pub. Date: Aug. 6, 2020

(54) DELAYED ONSET CALCIUM ALGINATE GELS AND METHOD OF PRODUCTION

(71) Applicant: Platform Biopolymer Technologies, LLC, Naples, FL (US)

(72) Inventors: Tycho Joseph Speaker, Santa Cruz, CA (US); W. Preston Brawn, Bradenton, FL (US)

(73) Assignee: Platform Biopolymer Technologies, LLC, Naples, FL (US)

(21) Appl. No.: 16/636,242

(22) PCT Filed: Jul. 25, 2018
(86) PCT No.: PCT/US2018/043655

§ 371 Date: Feb. 3, 2020

Related U.S. Application Data
(60) Provisional application No. 62/540,110, filed on Aug. 2, 2017.

(51) Int. Cl. C08J 3/075; C08L 5/04; A61L 9/04; C08K 5/098

(52) U.S. Cl. CPC C08J 3/075; C08L 5/04; C08J 2305/04; C08K 5/098; A61L 9/048

(57) ABSTRACT

A method of producing a gel comprises combining an acidic aqueous alginate solution with a second, less acidic aqueous suspension of a calcium salt. A product comprises an alginate-based air treatment gel that is not subject to temperature-induced liquefaction.

This application claims priority to U.S. Provisional Application No. 62/540,110, filed 2 Aug. 2017, the entire disclosure being incorporated herein by reference in its entirety.

### FIELD OF THE INVENTION

This invention relates generally to aqueous gels for the controlled release of fragrance, commonly called air treatment gels or air fresheners. It further relates to systems designed to prevent settling or creaming of suspended droplets or particles and to related products and processes for making and using such products. The invention further relates to manufacture of rheologically useful aqueous systems by providing a system wherein a triggerable interaction between two components produces a controlled thickening and gelation of the systems. The invention further relates to gels formed by the cross-linking of alginic acid and/or salts thereof and calcium ions.

### BACKGROUND OF THE INVENTION

The use of aqueous polymer gels as air freshener products is well-established and a substantial commercial market exists for such products. Numerous technologies have been developed to produce such gels, and the most widely used of these rely upon thermoreversible gelation, in which a material that is liquid at elevated temperature is hot-filled into a package that serves as a mold. Upon cooling, the material gels such that at least part of the package can be removed, exposing the material to ambient air such that a fragrance component can evaporate as a method of air treatment. Heating of the thermoreversible gel is an energy intensive step, adding to the cost of a generally low-priced commodity item. Compounding the energy costs, as a practical matter of production, relatively inefficient forced air cooling is normally used to speed the gelation process, to minimize the amount of product volume in the automated manufacturing production line. Further, heating to liquefy thermoreversible gels promotes both evaporative loss and degradation of fragrance components, adding cost and limiting or precluding the use of heat-labile fragrance components. Lastly, liquefied thermoreversible gels will rapidly solidify in nozzles and transfer lines if the manufacturing line is stopped, necessitating even more costly delays in production while lines and nozzles are cleaned or replaced. Therefore an unmet need exists for fast-setting aqueous gel systems that may be transferred in liquid form without concerns of premature gelation, and even further need exists for such gel systems that may be produced without expensive heating and cooling steps. The present invention describes methods and compositions of producing such aqueous gels that set rapidly from a liquid state without the use of heating or cooling processes.

### SUMMARY OF THE INVENTION

The invention provides a method of producing a gel, comprising combining an acidic aqueous alginate solution with a second, less acidic aqueous suspension of a calcium salt.

The acidic aqueous alginate solution may have a pH between 4 and 5.

The suspension of calcium salt may have a pH between 5 and 8.

The combined solution and suspension may have a pH between 4 and 6 immediately after mixing.

The calcium salt may be at least 10 times more soluble at the pH immediately after mixing than in the aqueous suspension.

The acidic aqueous alginate solution may comprise an acidic pH modifier, preferably selected from the group consisting of acetic acid, citric acid, phosphoric acid, hydrochloric acid, sulfuric add, and lactic acid, malic acid, and combinations thereof. The calcium salt suspension may further comprise a suspending agent, preferably selected from the group consisting of acacia gums, agar, acrylic acid, albumins, carrageenans, carbopols, casein, cellulose gums, chitosan, chondroitin, curdian, gelatin, dextran, fibrin, fulcelleran, gellan gum, ghatti gum, guar gum, gum tragacanth, heparin, hyaluronic acid, karaya gum, locust bean gum, pea protein, pectin, polyoxyethylene-polyoxypropylene and other synthetic block copolymers, pullulan, starch, soy protein, whey protein, xanthan gum, and zein, and combinations thereof. The calcium salt suspension may further comprise a basic pH modifier, preferably selected from the group consisting of soluble hydroxides, bicarbonates, amines, or similar bases soluble in water, and combinations thereof.

At least one fragrance may be dissolved, dispersed, or entrapped in the resulting gel. The fragrance may be dispersed or solubilized by the inclusion of at least one surfactant, preferably selected from the group consisting of ethylene oxide (ethoxylate) derivatives of alkanes, alcohols, fatty acids and other species including polysorbates, oleths, steareths and related forms, alkyl poly glycosides and related species, glucosides, esters of sorbitan and glycerol, alkyl amines, amides and related species, glutamates and related species, sulfates, sulfonates, sulfoacetates, phosphates and related species, and combinations thereof.

The fragrance dispersion may further comprise at least one cosolvent, preferably selected from the group consisting of alkanes, alcohols, amides, ethers, esters, glycols, and combinations thereof.

Additional materials may be entrapped or encapsulated within the gel as formed.

The alginate solution and the suspension of calcium salt may be produced by combining water with pre-blended dry powder, solid, capsule, tablet, granulate, paste, or concentrated forms of the component materials.

The invention also provides a product comprising an alginate-based air treatment gel that is not subject to temperature-induced liquefaction.

The product may exclude thermally reversible gel components, for example carrageenan, agar, or thermally reversible hydrocolloid gel combined systems. The product may further comprise a calcium salt component having a solubility of less than 5% at or above pH 7 but which is 95% or more soluble at a pH of 3-7. The product may exclude delta gluconolactone.

The product may exclude carboxymethylcellulose.

The product may be made by any of the methods recited above.

The product may contain citrate ion in an amount at least 100% equivalent to the amount of calcium ion present, calculated on a tricalcium citrate basis.

### DETAILED DESCRIPTION OF THE INVENTION

The invention provides a composition including an alginic acid component consisting of alginic acid and/or one or more salts thereof, and a calcium ion component wherein the calcium ion is present in an amount in a range from 0.1 wt % to 1000 wt % relative to the alginic acid component, both on a dry basis. The invention further provides a stable two-part system that upon combining the parts, produces a stable gel of a preselected strength. The invention further provides a composition that remains liquid after combination of the parts for a time sufficient to transfer the composition to a form such that the gelation occurs after transfer of the material, avoiding blockage of dispensing apparatus and other problems associated with immediate gelation. The invention further provides a method of producing such gels by combining said components.

The inventors combined an acidified sodium alginate solution with a second solution containing locust bean gum and suspended tricalcium citrate powder, with the expectation that the rapid dissolution of calcium ions in the resulting acidic solution would produce near-instantaneous gelation due to the rapid cross-linking reaction commonly used to produce calcium alginate gels. Unexpectedly, the resulting combined solution remained liquid and pourable for well over 1 minute, but within 3 minutes set to a firm gel that retained its shape upon inversion of the container.

As the gels of the present invention may be used as reservoirs for dissolved or dispersed fragrance in air treatment gels, an important aspect of such applications is that the gels are exposed to air in use and may be packaged such that in some cases a vertical surface of gel is exposed unsupported by any packaging, or in some cases the gel is exposed to air as an essentially free-standing composition. It is an important quality of such gels that they do not spontaneously flow or sag under such conditions, which might lead to unintended contact of dissolved fragrances with underlying surfaces, for example furniture or pianos.

Unless otherwise indicated, all percentages recited herein are on a weight basis.

Alginate

As used herein, unless otherwise or more precisely specified, “alginate” refers to alginic. acid, its ionized water-solvated form, salts thereof, or any of these chemical species that have been chemically modified by processes including partial hydrolysis, esterification, derivatization, and other methods, but retain the backbone structure of repeating glucuronic and mannuronic acid units that are defining of the alginic acid molecule and sufficient calcium-binding sites to form a gel in the presence of calcium. The mannuronic content of the alginate on a weight basis is typically at least 5%, or at least 25%, or at least 50%, or at least 75%, or at least 95%.

The viscosity of a 1.0% aqueous solution of the alginate as the sodium salt is typically at least 5 centipoise, or at least 50 centipoise, or at least 500 centipoise. It is typically at most 10 centipoise, or at most 100 centipoise, or at most 1000 centipoise.

Useful concentrations of alginate for the purposes of the invention may typically be at least 0.1%, 0.2%, 0.4%, 0.6% or 1.0%. They may typically be at most 10%, 5%, 2%, 1% or 0.5%.

Preparing Compositions of the Invention

Methods of forming gelled aqueous materials according to the invention will now be described in detail, followed by a description of suitable materials for use in making the solutions and materials.

Formation of Delayed-Onset Gels

The essential elements required to form the delayed-onset gels of the present invention are as follows:
1) An aqueous solution (phase A) of alginic acid and/or its water-soluble salts is prepared and may be further adjusted to a preselected pH lower than pH 7 (neutral)
2) An aqueous suspension (phase B) of a calcium salt (or salts) is prepared at a preselected pH that is higher than the pH of solution A and sufficiently high that the majority of the calcium salt component remains substantially (more than 95%) insoluble but the salt(s) is/are soluble at a pH between 3 and 7, for example a pH of 6.0.
3) Components A and B are combined with agitation to mix, with the resultant composition having a pH of about 6 or lower. The formation of a cross-linked calcium alginate gel occurs after mixing.

The respective solutions may further comprise additional polymer or other dissolved or suspended components, if a polymer is used to facilitate suspension of the calcium salts of suspension B, such suspending polymers must be selected such that suspension B is not gelled by the interaction of the polymers with the suspended calcium salt(s).

The properties of the resulting gel and the time to gelation are found to be controlled by multiple factors, including:
1) Selection of the alginate component(s)
2) The pH of the respective solutions
3) The concentrations of the alginate component(s)
4) The selection of the calcium salt(s)
5) The use level of the calcium salt(s)
6) The selection and use level of other components in either phase A or B.

Precursor Alginate Solution Phase A

A range of alginate solution concentrations may be used according to the invention, and the particular range is specific to the material being used. Alginates for instance may be produced in a wide range of viscosities, this quality being dependent upon structure and molecular weight. Commercial alginates are also graded as to the ratio of glucuronic to mannuronic acid units, also related to viscosity. Without wishing to be bound to any particular conceptual framework, generally, solutions of higher glucuronic acid content alginates exhibit stronger gel development in the invention, Thus correspondingly lower concentrations are required to achieve similar gel strength. For example an aqueous composition comprising 1% wt sodium alginate (Manucol DH, FMC Health & Nutrition, Philadelphia, Pa.) at pH 4.5 combined with a 0.1% suspension of tricalcium citrate (TCC) (Sigma-Aldrich, St. Louis, Mo.) in 1% locust bean gum (ESP) at pH 7, forms a non-sagging gel suitable for use as an air treatment gel. Without wishing to be bound to any particular conceptual framework, use of an alginate with a higher glucuronic to mannuronic acid content ratio (such as Manugel GHB, FMC Health & Nutrition, Philadelphia, Pa.) would produce similar rheological systems at a relatively lower concentration.

Phase A may further comprise a succulent extract, for example, aloe extract, as taught in PCT WO2016168179A1 “SUCCULENT EXTRACT AND ALGINATE COMBINED SOLUTIONS AND PRODUCTS INCORPORATING THEM” (Speaker and Brawn), incorporated herein by reference. It is a requirement that to the extent that additional components in the precursor alginate solution may influence or inhibit gelation at low calcium concentrations, such components are not present at levels so high that gelation is prevented after the components are combined.

Control of pH in Phase A

A wide variety of materials are well-known and understood to be useful in modifying the pH of aqueous solutions, and any of these materials are considered useful in the present invention. The pH of the alginate solution phase A is maintained below pH 7, or below pH 6 or 5, and most preferably at about pH 4. Citric acid is a preferred material for controlling pH in the present invention due to its low cost, safety, ease of handling, and wide availability from a range of sources, including natural and renewable sources. Other exemplary acids useful in the present invention include but are not limited to acetic acid, phosphoric acid, hydrochloric acid, sulfuric acid, and lactic acid, malic acid, or combination thereof, but generally any acid or combination of acids is considered useable in the present invention. The acidifying pH modifiers may typically comprise at least 0.01%, 0.05%, 0.1%, 0.5%, 1%, or 5% of the precursor solution in total weight. They may typically comprise at most 5%, 1%, 0.5%, 0.1%, or 0.05% by total weight.

Suspension of Calcium Salts in Phase B

In order to produce uniform distribution of the calcium salts that dissolve to form the gels of the present composition, it is desirable to maintain these in a suspension that maintains homogeneity over a useful time period. A variety of materials in aqueous solution can suspend calcium salts, and any material that is not itself reactive with calcium salts or ions is potentially useful for this purpose. In particular, in order to make the compositions of the present invention in an economical fashion, materials that provide good performance as suspending agents but are inexpensive at useful levels, safe, and ideally widely available from natural and/or renewable sources are most desirable. A wide variety of gums and polymers are available commercially, many of which may be used, singly or in combination including but not limited to, acacia gums, agar, acrylic acid, albumins, carrageenans, carbopols, casein, cellulose gums, chitosan, chondroitin, curdian, gelatin, dextran, fibrin, fulcelleran, gellan gum, ghatti gum, guar gum, gum tragacanth, heparin, hyaluronic acid, karaya gum, locust bean gum, pea protein, pectin, polyoxyethylene-polyoxypropylene and other synthetic block copolymers, pullulan, starch, soy protein, whey protein, xanthan gum, and zein. In a preferred embodiment of the present invention Locust bean gum (LBG) usefully meets the above criteria and further exhibits a degree of synergistic thickening with alginates, further strengthening the resulting gels. In another preferred embodiment, cornstarch may be used in place or in addition to LBG.

Tricalcium citrate is commonly available as a very fine powder that is very easily and stably suspended in the locust bean gum. This is advantageous in terms of not having to maintain suspension by constant stirring or other means. Grinding or otherwise producing finely divided forms of other calcium salts described produces forms suitable to provide similar ease of suspension.

Phase B may comprise a succulent extract as taught in PCT WO2016168179A1 (Speaker and Brawn). It is a requirement that to the extent that additional components in the phase B may influence or inhibit gelation at low calcium concentrations, such components are not present at levels so high that gelation is prevented after the components are combined.

Selection and Control of pH in Phase B

It is a requirement that the pH of phase B must be sufficiently high to prevent complete dissolution of the calcium salt component. Some polymers that might be selected to maintain calcium salts in suspension as described below might acidify an aqueous solution, and to prevent premature dissolution of the calcium salts, an additional pH modifier can be used to raise the pH of phase B to an appropriate level to prevent such dissolution. Generally any basic material can be used to this purpose, but exemplary materials include soluble hydroxides, bicarbonates, amines, or similar bases soluble in water, or combinations thereof. The basifying pH modifiers may typically comprise at least 0.01%, 0.05%, 0.1%, 0.5%, 1%, or 5% of the precursor solution in total weight. They may typically comprise at most 5%, 1%, 0.5%, 0.1%, or 0.05% by total weight. The pH of phase B may typically be at least 7.1, or at least 7.3, 7.5, or 8.0. It may typically be at most 9.5, or at most 9.0.

Formation of Gelled Materials

Gelled materials may be formed or cast by combining an aqueous precursor solution, A, for example 1% aqueous sodium alginate (Manucol DH, FMC) solution adjusted to pH 4 with citric acid, with a second liquid, B, comprising a suspension of a calcium salt, for example tricalcium citrate (Jungbunzlauer, Inc., Newton Centre, Mass.), at pH 7 suspended in a 1% solution of LBG (ESP). When the component liquids are combined, acidification of the suspended calcium results in the rapid dissolution of the calcium salt to produce calcium ions, which induce cross-linking gelation of alginate solutions. The liquids mix smoothly and uniformly, and remain pourable for typically about 1 m, beyond which rapid thickening is observed. If the system is left undisturbed for approximately an additional 2 m, it forms a gel that may be inverted and removed from the container without loss of its shape, indicating full solidification. After 12 h the same system can be stored indefinitely as a free-standing solid without observable sagging or flow, although evaporative loss will result in slow, contractive collapse.

Without wishing to be bound by any particular model, the strength and rigidity of the gel is understood to increase with increasing concentration of each of the solutions.

Selection of solution concentrations, pH, and particular alginates and calcium sources influences gel strength and the speed with which the gel forms.

US Patent Application 2005/0037080A, AIR TREATMENT GEL AND METHOD FOR ITS PREPARATION (Lynch et al) discloses air treatment gels formed by combining solutions of a gelling polymer (alginate), a gelling agent (calcium salts), and a non-gelling polymer (carboxymethylcellulose), and subsequently combining them with a pH modifier. Lynch's system differs from the present one in some important aspects. For example Lynch et al explicitly teach that use of organic acids, such as citric acid, lactic acid and acetic acid, that reduce pH quickly when added to such systems results in poor functionality, inhomogeneity, gels of insufficient rigidity, and/or excessive syneresis (shedding of liquid at the surface of the gels). Surprisingly, in the present invention, these materials combined effectively to produce gels of good strength with no observable syneresis.

Lynch et al further disclose that carboxymethylcellulose (CMC) is particularly well-suited as a non-gelling polymer component in the systems they describe, having very positive effects on storage stability and in particular syneresis, while not degrading gel strength or integrity. Very surprisingly, in the present invention CMC tested, including AkzoNobel AF 1985, AF 2805, AF 2785 and other grades, Ashland Chemical Aqualon grades, and CPKelco Finnfix grades, was found to substantially degrade gel strength, rendering the resulting gels insufficiently strong to remain unsupported without flowing and puddling, even after 48 h curing time. In contrast, LBG was found to be effective, and maintained functional suspension of calcium salts at substantially lower concentrations than any of CMC components tested, CMC was therefore not found to be useful in the role of a primary non-gelling polymer component in the present invention. Combination of phases A and B may be accomplished by any of the many known process by which liquids are combined. Without limitation, exemplary processes include addition of one phase to the other while stirring or mixing by other means, by co-injection through a static mixer, by centrifugal mixing, by co-spraying, by injection of one phase into the other under pressure to induce mixing, and any other process by which liquids may be reasonably rapidly mixed. In order to form a homogenous gel of maximal strength, the mixing of the two phases must be fully accomplished before the resulting mixture forms a solid gel.

Rigid gels may also be formed by deposition of alternating layers or compartments of phase A and B in sufficiently small volumes or layers to permit diffusion of calcium ions into adjacent layers, in a manner conceptually similar to a laminated material. Thus, accumulation of droplets of each of the phases will create a gel similar to the homogenous gel accomplished by the more complete mixing afforded by stirring the two phases together, larger droplets or layers incur calcium diffusion over longer distances, and thus an associated slowing of the overall gelation process. Gels produced according to the invention may typically contain citrate ion in an amount approximately equivalent to the amount of calcium ion present, calculated on a tricalcium citrate basis. The citrate ion content may be at least 50%, or at least 60%, 70%, 80%, 90%, 95%, or 100% of the equivalent amount. It may typically be at most 300%, or at most 250%, 200%, 150%, or 125% of the equivalent amount. In this context, “citrate ion” includes the ion in all forms, either free or as a salt, and at all degrees of protonation. On the other hand, the gels may typically contain less than an approximately equivalent amount in total of chloride, sulfate, and acetate ions, calculated on the basis of the corresponding calcium salts. The total amount of these ions may typically be less than 110%, or less than 100%, 90%, 80%, 70%, 60%, or 50% of the equivalent amount.

Gels produced according to the invention are typically not subject to temperature-induced liquefaction.

Additional Materials in Gels

The compositions may further comprise a dissolved or dispersed material suspended therein, for example a liquid, gaseous, or solid particulate material. The dispersed material may be partially or fully insoluble in the continuous phase, or may be complex materials such as microcapsules or cells. The additional materials may be initially dispersed in phase A, or they may be initially dispersed in phase B. Additional materials may be dispersed in both phases prior to combination, and in yet other cases the additional materials may be initially separate from both phases but introduced during mixing of the phases to form a gel.

The dissolved or dispersed material may be homogeneously distributed, or it may be inhomogeneously distributed. Or, one or more such materials may be homogeneously distributed while additional material(s) may be inhomogeneously distributed. The primary constraints upon what may be dispersed within the gels are that such materials may not destabilize the gels by disruption of the calcium alginate matrix, nor must it either inhibit gelation or trigger premature gelation in the precursor liquids. Therefore materials that scavenge calcium ions, or that produce highly oxidative or pH conditions not conducive to stable gels, may not reasonably be included in the present invention. However, materials that are largely inert in regard to gel destabilizing effects may readily be dispersed, or if the materials present a larger volume than the gels, the gels may be formed interstitially in the manner of mortar filling the space between stones. Additional components may comprise fragrances and may further comprise surfactant materials and/or cosolvents, and in particular those that are useful in dispersing or solubilizing fragrances, as are commonly used to incorporate fragrance into aqueous systems. Many solubilizers are known, including but not limited to ethylene oxide (ethoxylate) derivatives of alkanes, alcohols, fatty acids and other species including polysorbates, oleths, steareths and related forms, alkyl poly glycosides and related species including but not limited to glucosides, esters of sorbitan and glycerol, alkyl amines, amides and related species including but not limited to glutamates and related species, sulfates, sulfonates, sulfoacetates, phosphates and related species, and generally any material with surfactant properties useful for this purpose or combinations thereof. Cosolvents include, but are not limited to alkanes, alcohols, amides, ethers, esters, glycols, and other common materials useful to the purpose or combinations thereof. These materials may typically constitute at least 0.1%, 0.5%, 1%, 5%, 10% or 50% of the precursor solution in total weight. They may typically constitute at most 75%, 50%, 25%, 10%, or 5% by total weight.

Additional components may comprise materials that provide a visual effect, such as pigments, dyes, and other colorants or suspended particles. These materials may comprise the rehydratable gel particles, beads, or shaped gels taught in PCT WO2016168179A1, (Speaker and Brawn). They may typically constitute at least 0.1%, 0.5%, 1%, 5%, 10% or 50% of the precursor solution in total weight. They may typically constitute at most 75%, 50%, 25%, 10%, or 5% by total weight.

The additional components may comprise a succulent extract as taught in PCT WO2016168179A1 (Speaker and Brawn). R is a requirement that to the extent that additional components in the precursor alginate solution may influence or inhibit gelation at low calcium concentrations that such components are not present at levels so high that gelation is prevented after the components are combined.

Entrapment of Material in Gels

Materials may be entrapped in or encapsulated by the gels thus produced. Generally materials that are suitable for entrapment meet the same requirements as those suitable for dispersion in the gels. The gel may comprise a larger volume surrounding the entrapped material, in the case of a particle suspended in a volume of gel, while in other cases the gel may comprise a smaller total volume than the entrapped particle, as in the case of a thin layer of gel surrounding a larger particle. Entrapped payload materials may typically constitute at least 0.01%, 0.1%, 1%, 10% or 25% by weight of the droplet composition. The entrapped payload materials may typically constitute at most 99%, 90%, 75%, 50% or 30% by weight of the composition. p Limitations on included Components

Some materials can potentially interfere with gel formation or induce premature gel formation in at least one phase of the present invention and it therefore may be necessary to exclude or limit such material in some embodiments.

Antiperspirants, for example aluminum chlorohydrate and other aluminum compounds with similar activity, may be excluded from the composition, or these materials may be tolerated as long as the amount, relative to alginate, is no more than 5%, 1%, 0.5%, 0.1%, or 0.05%.

Antifungal or antidandruff agents, including zinc pyrithione and other zinc compounds with similar activity may be excluded from the composition, or these materials may be tolerated as long as the amount, relative to alginate, is no more than 5%, 1%, 0.5%, 0.1%, or 0.05%.

Quaternary amines may be excluded from the composition, or these materials may be tolerated as long as the amount, relative to alginate, is no more than 5%, 1%, 0.5%, 0.1%, or 0.05%,

Formation of Gels of Particular Shapes or Dimensions

The compositions of the present invention are initially liquids after the component phases A and B are combined, and as liquids will fill a portion of any container into which they are introduced under the influence of gravity, or other acceleration such as centrifugation for example. As the transient liquid mixture promptly solidifies to a gelled state, the resulting gel can be thus molded, retaining the shape under which it initially solidified. Essentially any shape can be formed by the compositions of the present invention that can be formed by other cast-molding methods well known to those of ordinary skill in a wide variety of industries in which shapes are thus formed. The compositions of the present invention can be used to form a gel that further comprises one or more suspended components, and then subsequently to form an additional gel deposited on top of the first that comprises a different suspended component or components, to form a visibly distinct layer. For example such layers can comprise one or more colorants. It will be obvious to a person of ordinary skill in the manufacture of air treatment gels that many visually attractive and interesting layered compositions may be thus formed. Further, component gels can be cast in recognizable shapes, for example flowers, or radial stripes, and a second gel can be deposited over these in a contrasting color filling the spaces left when the primary mold was removed. The gels can be formed so as to include a void space, or may be applied or deposited so as to form a coating on a surface or object.

Dry Powders for use in the Present Invention

It will be apparent to persons of ordinary skill in making and acidifying alginate solutions that a particular alginate component may be pre-blended in dry or suspended form with a particular acid or acidic components such that upon with an appropriate quantity of water, solutions useful as phase A of the present invention can be readily produced. Thus precursor phase A can be provided in a commercially convenient dry powder form. Similarly, a solid, capsule, tablet, granulate, paste, concentrate or other form suitable for dissolution in water at the point of use is readily produced.

Just as the alginate phase A can be produced in dry or concentrated form, the components of phase B can similarly be produced in a commercially convenient form for addition to water to form phase B with preselected levels of each component as taught in the present invention.

### EXAMPLES

In some aspects, the invention provides a method of making aqueous gels, by combining two component phases, one of which comprises an acidic alginate solution, and the other of which comprises a dispersed calcium salt and may further comprise suspending agents, and in which the calcium salt is not completely solubilized. In some aspects, the invention provides a product comprising the two precursor phases comprising respectively the alginate phase and the calcium suspension phase. In some aspects, the invention provides a product formed by the combination of the two phases to form a gel.

In some aspects, the invention provides a product comprising an air treatment gel and further comprising a succulent extract complexed with an alginate.

### Example 1

### Formation of a Gelled Aqueous System

An aqueous solution, A, comprising 50 mL of 2.0% wt. sodium alginate (Manucol DH, FMC Health & Nutrition, Philadelphia, Pa.) was prepared, and adjusted to pH 4.0 by addition of, approximately 0.15 mL of a 10% aqueous solution of citric acid (ESP). A second aqueous solution, B, comprising 50 mL of 1.0% wt. Locust Bean Gum (ESP) and further comprising 0.1 g tricalcium citrate (ESP) in suspension was added to solution A while it was stirred at 700 rpm. The resulting solution thickened but remained a pourable liquid for over 60 s. At approximately 120 s after addition of solution B, the magnetic stir bar stopped spinning despite the action of the underlying stir plate, due to solidification of the system. At approximately 180 s, the beaker was removed from the stir-plate and inverted on the adjacent countertop and no flow or movement, of the thus-formed gel was observed, nor did the gel flow or migrate from the beaker upon standing overnight at room temperature.

### Example 2

### Formation of an Air-Treatment Gel by Co-Extrusion of Phases

A 20 mL aliquot of each of the precursor phases A and B taught in Example 1. was prepared, and to phase A was further introduced 2.5 mL of a commercial fragrance (Citrus Pine Diffuser, Robertet USA, Mt. Olive, N.J.). These phases were loaded into respective chambers of a dual-barrel dispensing syringe fitted with an inline static mixer(Perigee Epoxy Supply, North Richland Hills, Tex.), and the plunger was depressed, dispensing the two phases through the inline static mixer tube into a weighing boat. The liquid mixture thus formed flowed evenly into the weighing boat and formed a flat, level layer in the manner of moderate viscosity liquids introduced to a container. After 3 minutes, the weigh boat was inverted and the gel formed in situ showed no evidence of sagging or flow, indicating satisfactory set suitable for an air treatment gel. The resulting gel demonstrated roughly 7% loss per day of air exposure at room temperature, and was effective at providing fragrance in ambient air.

### Example 3

### Formation of an Air-Treatment Gel including a Succulent Extract

An aqueous solution, A, comprising 20 mL of 1.0% wt, sodium alginate (Manucol® DH, FMC Health & Nutrition, Philadelphia, Pa.) combined with 20 mL of a 1.0% solution of Aloe vera inner leaf powder (ESP) was prepared, and adjusted to pH 4.0 by addition of, approximately 0.05 mL of a 10% aqueous solution of citric acid (ESP). The final phase A assumed shear-thinning gel rheological properties typical of such succulent/alginate systems. To phase A was further introduced 2.5 mL of a commercial fragrance without surfactant solubilizers, which after mixing remained suspended in the fluid gel rheological system provided by phase A. A second aqueous solution, B, comprising 50 mL of 1.0% wt. Locust Bean Gum (ESP) and further comprising 0.1 g tricalcium citrate (ESP) in suspension was added to solution A while it was stirred at 700 rpm. The resulting solution thickened but remained a pourable liquid for over 60 s. At approximately 120 s after addition of solution B, the magnetic stir bar stopped spinning despite the action of the underlying stir plate, due to solidification of the system. At approximately 180 s, the beaker was removed from the stir-plate and inverted on the adjacent countertop and no flow or movement of the thus-formed gel was observed, nor did the gel flow or migrate from the beaker upon standing overnight at room temperature.

1. A method of producing a gel, comprising combining an acidic aqueous alginate solution with a second, less acidic aqueous suspension of a calcium salt.

2. The method of claim 1 wherein the acidic aqueous alginate solution has a pH between 4 and 5.

3. The method of claim 1, wherein the suspension of calcium salt has a pH between 5 and 8.

4. The method of claim 1, wherein the combined solution and suspension has a pH between 4 and 6 immediately after mixing.

5. The method of claim 1, wherein the calcium salt is at least 10 times more soluble at the pH immediately after mixing than in the aqueous suspension.

6. The method of claim 1, wherein the acidic aqueous alginate solution comprises an acidic pH modifier, preferably selected from the group consisting of acetic acid, citric acid, phosphoric acid, hydrochloric acid, sulfuric acid, and lactic acid, malic acid, and combinations thereof.

7. The method of claim 1, wherein the calcium salt suspension further comprises a suspending agent, preferably selected from the group consisting of acacia gums, agar, acrylic acid, albumins, carrageenans, carbopols, casein, cellulose gums, chitosan, chondroitin, curdlan, gelatin, dextran, fibrin, fulcelleran, gellan gum, ghatti gum, guar gum, gum tragacanth, heparin, hyaluronic acid, karaya gum, locust bean gum, pea protein, pectin, polyoxyethylene-polyoxypropylene and other synthetic block copolymers, pullulan, starch, soy protein, whey protein, xanthan gum, and zein, and combinations thereof.

8. The method of claim 1, wherein the calcium salt suspension further comprises a basic pH modifier, preferably selected from the group consisting of soluble hydroxides, bicarbonates, amines, or similar bases soluble in water, and combinations thereof.

9. The method of claim 1, wherein at least one fragrance is dissolved, dispersed, or entrapped in the resulting gel.

10. The method of claim 1, wherein fragrance is dispersed or solubilized by the inclusion of at least one surfactant, preferably selected from the group consisting of ethylene oxide (ethoxylate) derivatives of alkanes, alcohols, fatty acids and other species including polysorbates, oleths, steareths and related forms, alkyl poly glycosides and related species, glucosides, esters of sorbitan and glycerol, alkyl amines, amides and related species, glutamates and related species, sulfates, sulfonates, sulfoacetates, phosphates and related species, and combinations thereof.

11. The method of claim 1, wherein fragrance dispersion further comprises at least one cosolvent, preferably selected from the group consisting of alkanes, alcohols, amides, ethers, esters, glycols, and combinations thereof.

12. The method of claim 1, wherein additional materials are entrapped or encapsulated within the gel as formed.

13. The method of claim 1, wherein the alginate solution and the suspension of calcium salt are produced by combining water with pre-blended dry powder, solid, capsule, tablet, granulate, paste, or concentrated forms of the component materials.

14. A product comprising an alginate-based air treatment gel that is not subject to temperature-induced liquefaction.

15. The product of claim 14, wherein the product excludes thermally reversible gel components, for example carrageenan, agar, or thermally reversible hydrocolloid gel combined systems.

16. The product of claim 14, further comprising a calcium salt component having a solubility of less than 5% at or above pH 7 but which is 95% or more soluble at a pH of 3-7.

17. The product of claim 14, wherein the product excludes delta gluconolactone.

18. The product of claim 14, wherein the product excludes carboxymethylcellulose.

19. The product of claim 14, made by the method of claim 1.

20. The product of claim 19 wherein the product contains at least contain citrate ion in an amount at least 100% equivalent to the amount of calcium ion present, calculated on a tricalcium citrate basis.
